(19) United States
(12) Patent Application Publication
(10) Pub. No.: US 2002/0001855 A1
(43) Pub. Date: Jan. 3, 2002

(54) Methods and apparatus for parallel magnetic biological analysis and manipulation

(76) Inventors: Mara G. Prentiss, Cambridge, MA (US); Gary Zabow, Somerville, MA (US)

Correspondence Address: Timothy J. Oyer, Wolf, Greenfield & Sacks, P.C., 600 Atlantic Avenue, Boston, MA, 02210, US

(21) Appl. No.: 09/815,255

(22) Filed: Mar. 22, 2001

Related U.S. Application Data
(60) Provisional application No. 60/191,392, filed on Mar. 22, 2000.

(51) Int. Cl. G01N 33/553

(52) U.S. Cl. 436/526

(57) ABSTRACT

The invention relates to a new method for conducting parallel manipulations of biological entities with emphasis on measurements of binding strengths between, or elastic strengths of, various biological molecules. In particular, the invention involves the generation and use of high magnetic fields and in particular high magnetic field gradients over extended areas using electromagnetic or permanent magnets in one- or two-dimensional grids, which can be used to measure simultaneously multiple binding interactions. The binding interactions may all be the same or may each be different from the others.

RELATED APPLICATIONS

This application claims priority to U.S. Provisional Patent Application Serial No. 60/191,392, filed Mar. 22, 2000, entitled “Methods and Compositions For Parallel Magnetic Biological Analysis”, the disclosure of which is incorporated herein by reference.

### FIELD OF THE INVENTION

This invention relates to a new method for conducting parallel manipulations of biological entities with emphasis on measurements of binding forces or strengths between, or elastic forces or strengths of, various biological agents.

### BACKGROUND OF THE INVENTION

With the advent of the human genome project and bioinformatics, high speed diagnostics are becoming very desirable. Diagnostic systems which are capable of parallel processing enable a multitude of tests to be performed simultaneously, and therefore reach high-through put capacity. Many currently available diagnostic systems are based on complex biological interactions and phenomena. Therefore, trouble shooting for errors in these systems is sometimes laborious, often limited to trial and error tests. Parallel processing is essential for the analysis of large numbers of biological elements which can currently be synthesized using combinatorial chemistry and library technology such as nucleic acids, polypeptides, lipids, carbohydrates, etc., because it facilitates much more efficient drug screening and development, faster mapping of biological functions.

An example of a parallel system capable of high throughput is DNA chip microarray analysis. In this type of analysis, microchips are designed in a grid pattern and each site within the grid can contain (i.e., present) a different biological entity. The biological entities which have been used successfully are generally nucleic acid in nature and include primers specific for a particular gene, or entire or partial genes. Although not currently available, such technology may also embrace biological entities such as peptides or polypeptides including antibodies, transcription factors, and signal transduction factors and entire cells or cell lysates. As an indication of the power and demand for such technology, the DNA microarray chip market is predicted to top one billion dollars within 5 years (Science, Aug. 6, 1999).

### SUMMARY OF THE INVENTION

The invention relates to a new method for conducting parallel manipulations of biological entities with emphasis on measurements of binding and/or adhesion forces between, or elasticities of, various biological agents. The terms “biological agents,” “biological molecules” and “biological entities” are used interchangeably herein. The invention provides, in one aspect, methods and apparatus for parallel measuring of specific and/or non-specific binding interactions between biological entities. In another aspect, the invention provides methods and apparatus for parallel measuring of the elastic properties and/or strengths of various polymers including DNA. According to the invention, such binding interactions and elastic properties are measured using a one- or two-dimensional magnetic grid, with a plurality of regions or areas. Each region or area may contain a biological entity different from that at other regions or areas, or alternatively, all regions or areas may contain the same biological entity. The latter configuration allows measurement of one binding strength or one elastic strength to be performed many times in parallel thereby providing an improved signal-to-noise ratio and better statistics on the particular measurement in a shorter period of time. The methods and apparatus of the invention are designed to be easily compatible with a number of developing technologies including DNA microarray chip technology.

The invention further provides methods and articles for producing large magnetic field gradients and associated forces on biological agents over extended areas. The magnetic field geometry and manipulating forces on the biological entities can be easily switched between a spatially uniform and a spatially periodically modulated force for use in different biological experiments.

In one aspect, the invention provides a method for measuring a binding strength between at least one binding member and a cognate receptor. As used herein, a binding member (i.e., a binding partner) and a cognate receptor refer to the two molecules of a binary molecular interaction. The terms “binding member” and “receptor” can be used interchangeably, such that a receptor may be a binding member and a binding member may be a receptor. As used herein, a cognate receptor refers to a receptor which specifically recognizes and binds to a binding member as compared to a non-specific binding interaction. In some embodiments, the receptor may be one which recognizes the binding member non-specifically. The method involves contacting a magnetically-labeled cognate receptor with an at least one binding member which is conjugated to a first surface, applying a magnetic field (and magnetic field gradient) and magnetic force to the magnetically-labeled cognate receptor by positioning the first surface in proximity to a second surface having a magnetic field and a magnetic field gradient, and determining whether the magnetically-labeled cognate receptor remains in contact with the at least one binding member. As used herein, a magnetically-labeled cognate receptor is a biological molecule that binds to a binding member and which is bound (i.e., conjugated) to a magnetic particle. Whether a magnetically-labeled cognate receptor remains in contact with the binding member can be determined by whether the magnetically-labeled cognate receptor becomes attached to the second surface. For example, where the magnetic field and related magnetic gradient and force is greater than the binding strength of the interaction between the binding member and the magnetically-labeled cognate receptor, then the receptor and its attached magnetic particle will be present on the second surface. If the magnetic field (and magnetic field gradient) and force is less than the binding strength, then the receptor will remain in contact with the binding member and will not be present on the second surface.

In a related aspect, the invention provides a method for measuring an elastic strength of a biological molecule. The method involves binding a biological molecule which is labeled (i.e., conjugated) with a magnetic particle at one end (i.e., a magnetically-labeled biological molecule) to a first surface, applying a magnetic field (and magnetic field gradient) and force to the biological molecule by positioning the first surface in proximity to a second surface having a magnetic field and a magnetic field gradient, and determining a distance between the magnetic particle and the first surface as a measure of the elastic strength of the biological molecule after the magnetic force has been applied. The maximum distance which can be achieved between the magnetic particle and the first surface is an indication of the elastic strength of the molecule. In another embodiment, the stretching of the molecule as a function of the applied magnetic force can be measured to yield more complete information on the behavior of the molecule under different stresses.

According to either of the afore-mentioned methods, the magnetic field and the magnetic field gradient are produced by an arrangement selected from the group consisting of an antiparallel adjacent array of permanent magnets, an antiparallel array of electric current carrying wires, a magnetic media with a one- or two-dimensional periodic pattern, a permanent magnet with a regularly embossed one or two dimensional surface structure, and a magnetizable material formed in a one or two dimensional grid, or with an embossed one- or two-dimensional surface structure in the presence of an externally applied uniform magnetic field.

In one embodiment, the first surface has an area of at least 100 μm². In an important embodiment, the first surface has an area of at least 1 mm². In yet a further embodiment, the first surface has an area of at least 1 cm². And in still a further embodiment, the first surface has an area of at least 100 cm². In one embodiment, the magnetic field (and corresponding magnetic field gradient) is approximately uniform throughout an area of the second surface. As used herein, “approximately uniform” intends a variation within 20%. In preferred embodiments, the variation is within 10% and in even more preferred embodiments, the variation is within 5%. In another embodiment, the method further comprises varying the magnetic field (and the corresponding magnetic field gradient) of the second surface. The magnetic field (and the corresponding magnetic field gradient) may be varied by changing a distance between the first surface and the second surface. The magnetic field (and corresponding magnetic field gradient) may be varied, according to another embodiment, by varying or changing an electric current that travels through the electric current carrying wires. In another embodiment, the magnetic field (and corresponding magnetic field gradient) may be varied by changing the externally applied uniform magnetic field.

In one embodiment, the at least one binding member (or the biological molecule) is at least 10, at least 100, at least 1000, at least 10,000, at least 100,000, or at least 1,000,000 binding members (or biological molecules). The binding strength or elastic strength measurements may be performed simultaneously. Preferably, parallel measurements performed simultaneously are all binding strengths measurements or all elastic strength measurements.

According to another embodiment, the first surface is comprised of a plurality of discrete regions. The plurality of discrete regions may be arranged in a regular structure, or alternatively, they may be arranged in an irregular structure. Examples of regular structures are one dimensional grids, two dimensional grids or “chessboard” patterns. In one embodiment, a different species of binding member (or biological molecules) is conjugated or bound to each of the plurality of discrete areas.

In one embodiment, the first surface is at least 1 μm, at least 10 μm, at least 20 μm, at least 100 μm, or at least 200 μm, from the second surface. Preferably, the first surface is flat. The first surface may be made of any material although attention should be given to magnetizable materials and the effects they may have on the magnetically-labeled biological molecules. Examples of some non-magnetizable surfaces which would be suitable include glass or plastic including polypropylene or polystyrene. The first surface may be but is not limited to a microscope slide. An adhesive material, preferably in the form of a film or a sheet or a strip, may be overlaid on the second surface. The adhesive material may or may not be removable from the second surface.

In another embodiment, the magnetic field gradient is at least 0.1 Tesla/mm. In still another embodiment, the magnetic field gradient is lower than 0.1 Tesla/mm. In another embodiment, the magnetic recording media includes but is not limited to magnetic tape and magnetic disk with either a one- or two-dimensional periodic magnetization pattern recorded on it.

According to another aspect of the invention, another method is provided for measuring a binding strength between at least one binding member and a cognate receptor. The method comprises contacting a magnetically-labeled cognate receptor with an at least one binding member conjugated (i.e., attached or bound) to a first surface, applying a magnetic field and force (and corresponding magnetic field gradient) to the magnetically-labeled cognate receptor by positioning the first surface in proximity to a second surface having a magnetic field (and corresponding magnetic field gradient), and determining whether the magnetically-labeled cognate receptor remains in contact with the at least one binding member or whether the magnetically-labeled cognate receptor is attached to the second surface. In accordance with the method, the second surface has an area of at least 100 μm² and a magnetic field magnitude produced (i.e., applied) is approximately uniform throughout planes parallel to the first surface and the second surface. In an important embodiment, the second surface has an area of at least 1 mm². In yet other embodiments, the second surface has an area of at least 1 cm², or at least 100 cm².

In a related aspect of the invention, a method is provided for measuring an elastic strength of a biological molecule. This latter method comprises binding a biological molecule which is labeled at one end with a magnetic particle to a first surface, applying a magnetic force to the biological molecule by positioning the first surface in proximity to a second surface having a magnetic field and force (and corresponding magnetic field gradient), and determining a distance between the first surface and the magnetic particle as a measure of the elastic strength of the biological molecule after the magnetic force has been applied. In accordance with the method, the second surface has an area of at least 1 mm², and a magnetic field magnitude produced (i.e., applied) is approximately uniform throughout planes parallel to the first surface and the second surface.

In another aspect of the invention, a method is provided for mixing magnetically-labeled biological molecules. The method includes transporting a first type and a second type of magnetically-labeled biological molecule in a fluid stream. The method further includes mixing the first type and a second type of magnetically-labeled biological molecule within the fluid stream by applying a magnetic force using an arrangement selected from the group consisting of an antiparallel adjacent array of permanent magnets, an antiparallel array of electric current carrying wires, a magnetic media with a one- or two-dimensional periodic pattern, a permanent magnet with a regularly embossed one or two dimensional surface structure, and a magnetizable material formed in a one or two dimensional grid, or with an embossed one or two dimensional surface structure in the presence of an externally applied uniform magnetic field.

In one embodiment, the method further comprises varying the magnetic field (and corresponding magnetic field gradient) of the second surface. The magnetic field (and corresponding magnetic field gradient) may be produced with an adjacent array of antiparallel permanent magnets, or with magnetic recording media comprising an array of antiparallel recorded magnetizations or with a, preferably single, permanent magnet with an embossed surface structure in the form of a one or two dimensional grid. The magnetic field (and corresponding magnetic field gradient) may be varied by changing a distance between the first surface and the second surface. In another embodiment, the magnetic field (and corresponding magnetic field gradient) is produced with an antiparallel array of electric current carrying wires. Accordingly, the magnetic force may be varied by a change in electric current. In another embodiment, the magnetic field (and corresponding magnetic field gradient) is produced by magnetizing a grid or embossed surface of magnetizable material in the presence of an externally applied uniform magnetic field. Accordingly, the magnetic force may be varied by changing the externally applied field.

In another embodiment, one-dimensional and two-dimensional arrays are formed using a permanent magnet with a surface which has been embossed (e.g., etched or carved) to give an array structure on the surface. In another embodiment, one-dimensional and two-dimensional arrays are formed using a magnetizable material which either has an embossed surface (as in the previous embodiment), or is simply a grid itself.

In one embodiment, the area of the first surface is at least 100 μm², or at least 1 mm², or at least 1 cm², or at least 100 cm². The first surface may be comprised of a plurality of discrete regions, of regular or irregular structure. In another embodiment, the magnetic field gradient is at least 0.001 Tesla/mm, at least 0.01 Tesla/mm or at least 0.1 Tesla/mm.

In yet another aspect, an apparatus is provided for measuring a biological parameter. The apparatus comprises a first surface comprised of a magnetic field (and corresponding magnetic field gradient) generated by an arrangement selected from the group consisting of a planar, adjacent, antiparallel array of a plurality of permanent magnets, or a planar, adjacent antiparallel array of magnetizations recorded onto a magnetic recording media, a permanent magnet with a regularly embossed one or two dimensional surface structure, and a magnetizable material formed in a one or two dimensional grid, or with an embossed one or two dimensional surface structure in the presence of an externally applied uniform magnetic field. The magnetic field (and corresponding magnetic field gradient) is periodically modulated over an area of the first surface and decays non-linearly with distance from the first surface. Each of the plurality of permanent magnets, magnetizations or embossed surface structures has an area of less than 1 cm². The magnetic recording media may be a magnetic disk or magnetic tape, but is not so limited.

In one embodiment, the biological parameter is a binding strength between a binding member and its cognate receptor. In another embodiment, the biological parameter is an elasticity strength of a biological molecule.

In one embodiment, the planar, adjacent, antiparallel array is a linear array. In one embodiment, the planar, adjacent, antiparallel array is a linear array and the first surface has an area of at least 2 cm². In another embodiment, the planar, adjacent, antiparallel array is a linear array and the first surface has an area of at least 2 cm² and less than 1000 cm². In one embodiment, the first surface has an area of less than 750 cm², or less than 650 cm². In yet another embodiment, the planar, adjacent, antiparallel array is a linear array and the apparatus further comprises a second surface. In another embodiment, the planar, adjacent, antiparallel array is a linear array and the apparatus further comprises a magnetic particle in contact with the first surface. And in yet further embodiments, the planar, adjacent, antiparallel array is a linear array and the plurality of permanent magnets or magnetizations or embossed surface structures is at least 10, at least 20, at least 30, at least 50 or at least 100.

In another embodiment, the planar, adjacent, antiparallel array is a two dimensional array. In one embodiment, the plurality of permanent magnets or magnetizations or embossed surface structures is at least 10, at least 1000, or at least 1,000,000. In one embodiment, the area of the first surface is at least 1 mm², at least 1 cm², or at least 100 cm². The apparatus may further comprise an adhesive material contacting the first surface. The adhesive material may or may not be removable from the first surface.

The invention provides a related apparatus in which the magnetic field (and corresponding magnetic field gradient) is approximately uniform, and the planar adjacent array is one-dimensional. In one aspect, an apparatus is provided for measuring a biological parameter comprising a first surface comprised of a magnetic field and a magnetic field gradient generated by a planar, adjacent, antiparallel array of a plurality of permanent magnets, or a planar, adjacent antiparallel array of magnetizations recorded onto a magnetic recording media, wherein the magnetic field is approximately uniform over an area of the first surface and decays non-linearly with distance from the first surface, and wherein each of the plurality of permanent magnets or magnetizations has an area of less than 1 cm². In one embodiment, the first surface has an area of at least 2 cm². In another embodiment, the planar, adjacent, antiparallel array is a linear array. In another embodiment, if the array is linear, then the plurality of permanent magnets or magnetizations is at least 50. In other important embodiments, the first surface has an area of less than 1000 cm², less than 750 cm², and less than 650 cm². In one embodiment, where the array is a linear array, the apparatus further comprises a second surface. And in still a further embodiment, where the array is linear, the apparatus further comprises a magnetic particle in contact with the first surface

The invention further provides an apparatus for measuring a biological parameter which comprises a first surface with a magnetic field (and corresponding magnetic field gradient) comprised of a first planar, antiparallel array of electric current carrying wires, wherein the magnetic field (and corresponding magnetic field gradient) is periodically modulated throughout an area of the first surface and decays non-linearly with distance from the first surface and wherein the first surface has an area of at least 100 μm². In one embodiment, the biological parameter is a binding strength between a binding member and a cognate receptor. In another embodiment, the biological parameter is an elastic strength of a biological molecule.

In one embodiment, the apparatus further comprises a second planar, antiparallel array of electric current carrying wires overlaid on and perpendicular to the first planar, antiparallel array. In another embodiment, the area of the first surface is at least 1 mm², or at least 1 cm². In an important embodiment, the area of the first surface is at least 2 cm². In a further embodiment, the area of the first surface is at least 100 cm². In yet further embodiments, the first surface has an area of less than 1000 cm², less than 750 cm², and less than 650 cm².

In yet another embodiment, the apparatus further comprises a second surface having a plurality of discrete regions. The first surface and the second surface may be separated by 1 μm, 2 μm, 5 μm, 10 μm, 100 μm, or more. In a further embodiment, a biological molecule may be bound (i.e., conjugated) to the second surface. The biological agent may be a binding member, or it may be a cognate receptor of a binding member, but it is not so limited. In another embodiment, the apparatus further comprises an insulating material.

The invention provides a related apparatus in which the magnetic field (and corresponding magnetic field gradient) is approximately uniform and the planar antiparallel array is one-dimensional. The apparatus provided is one for measuring a biological parameter comprising a first surface with a magnetic field and a magnetic field gradient comprised of a first planar, antiparallel array of electric current carrying wires, wherein the magnetic field is one-dimensional and approximately uniform throughout an area of the first surface and decays non-linearly with distance from the first surface and wherein the first surface has an area of at least 2 cm². In one embodiment, the first surface has an area of at least 100 cm².

In another embodiment, the first surface has an area of less than 1000 cm², less than 750 cm², less than 650 cm², and less than 500 cm². In one embodiment, the apparatus further comprises a second surface having a plurality of discrete regions. The first surface and the second surface may be separated by at least 1 μm. The second surface may be conjugated to a biological molecule. The biological molecule may be a binding member. The apparatus, in another embodiment, further comprises an insulating material. The biological parameter may be a binding strength between a binding member and its cognate receptor, or an elastic strength of a biological molecule, but is not so limited.

In yet a further aspect, the invention provides an apparatus for measuring a biological parameter comprising a periodic magnetic array with an area of at least 1 mm² in close proximity to at least one biasing magnetic field (and corresponding magnetic field gradient). In an important embodiment, the periodic magnetic array has an area of at least 2 cm². The magnetic array may be comprised of all the foregoing magnetic arrays including an array of permanent magnets, an array of electric current carrying wires, and an array of magnetizations recorded onto a magnetic recording material, and an embossed permanent magnet or magnetizable material in the presence of an externally applied uniform magnetic field. The biasing magnetic field is produced (i.e., generated) by one or more permanent magnets of area greater than the magnetic array, or by an additional arrangement of electric current carrying wires either separate from, or attached to, the array. The resultant magnetic field (and corresponding magnetic field gradient) in the plane of the periodic magnetic array has a regular periodic variation in field strength.

In one embodiment, the at least one permanent magnet is positioned at the side of, or below, the periodic magnetic array. In one embodiment, the periodic magnetic array is comprised of a planar, antiparallel array of permanent magnets. In another embodiment, the periodic magnetic array is comprised of an first planar antiparallel array of electric current carrying wires overlaid on and perpendicular to a second planar antiparallel array of electric current carrying wires.

In another aspect, an article is provided comprising a one- or two-dimensional antiparallel array of magnets defining a surface comprised of alternating magnetic poles, the surface having an area of at least 1 mm². In an important embodiment, the surface has a area of at least 2 cm². In one embodiment, the antiparallel array of magnets is comprised of a magnetic recording material. In another embodiment, the antiparallel array of magnets is comprised of an array of permanent magnets. In yet a further embodiment, the antiparallel array of magnets is comprised of an array at least 10 magnets, preferably an array of at least 20 magnets and most preferably an array of at least 50 magnets. In another embodiment, the antiparallel array of magnets comprises an array of current carrying wires.

In another aspect, an article is provided comprising an antiparallel array of at least 10 magnets, preferably an array of at least 20 magnets and most preferably an array of at least 50 magnets. The antiparallel array may be comprised of magnetic recording material. The antiparallel array may also be comprised of an array of permanent magnets.

The invention further provides a method for measuring a plurality of biological strengths simultaneously comprising contacting at least one magnetically-labeled cognate receptor with a plurality of binding members conjugated to a first surface, applying a magnetic field and force (and corresponding magnetic field gradient) to the at least one magnetically-labeled cognate receptor by positioning the first surface in proximity to a second surface having a magnetic field (and corresponding magnetic field gradient), and determining whether each of the plurality of binding members remains in contact with the at least one magnetically-labeled cognate receptor or whether the magnetically-labeled cognate receptor is attached to the second surface for a given applied force. The magnetic field (and corresponding magnetic field gradient) is produced by an arrangement selected from the group consisting of an antiparallel adjacent array of permanent magnets, an antiparallel array of electric current carrying wires, a magnetic recording material with a one- or a two-dimensional periodic pattern, a permanent magnet with regularly embossed one or two dimensional surface structure, and a magnetizable material formed in a one or two dimensional grid, or with an embossed one or two dimensional surface structure in the presence of an externally applied uniform magnetic field. In one embodiment, the plurality of binding members is comprised of different species of binding members. In another embodiment, the first surface is comprised of a plurality of discrete regions, which may have a regular or an irregular structure (i.e., pattern).

In one embodiment, a different species of binding member is conjugated (i.e., bound or attached) to each of the plurality of discrete regions. The plurality of discrete regions may be at least 2, at least 8, at least 25, at least 50, at least 100, at least 1000, at least 10,000, or at least 100,000. The area of each of the plurality of discrete regions may at least 1 μm². In a preferred embodiment, the area of each of the plurality of discrete regions is less than 10 mm². In other embodiments, the area of each of the plurality of discrete regions may be less than 1 μm². In one embodiment, at least 10 binding member molecules are conjugated (i.e., attached or bound) to each of the plurality of discrete regions.

In another aspect, a related apparatus is provided for measuring a plurality of elastic strengths simultaneously comprising binding (i.e., attaching or conjugating) a plurality of magnetically-labeled biological molecules to a first surface, applying a magnetic field and force (and corresponding magnetic field gradient) to the plurality of magnetically-labeled biological molecules by positioning the first surface in proximity to a second surface having a magnetic field (and corresponding magnetic field gradient), and determining the distance between the first surface and the magnetic particle of the magnetically-labeled biological molecule.

In a further aspect, the invention provides an apparatus comprising at least two wires each with terminals addressable electrically, going back and forth across each other. In a preferred embodiment, the at least two wires is two wires.

These and other aspects of the invention will be described in greater detail below. Throughout this disclosure, all technical and scientific terms have the same meaning as commonly understood by one of ordinary skill in the art to which this invention pertains unless defined otherwise.

### BRIEF DESCRIPTION OF THE DRAWINGS

FIGS. 1A and 1B are schematics of sample permanent magnet arrays, showing checkerboard pattern of alternating magnet directions. The figures are not to scale.

FIGS. 2A and 2B are schematics of sample permanent magnet arrays, showing a 1-dimensional stripe pattern of alternating magnet directions. The figures are not to scale.

FIG. 3 is a schematic of a sample electromagnetic array formed using current-carrying wires. The grid is formed from two wires only, each a single serpentine coil (i.e., heating coil pattern) with two wire-patterns at right angles to each other. The figure is not to scale.

FIG. 4 is a schematic showing the use of a magnet array to pull beads up off a bottom surface which has an array of different bio-coatings. In accordance with the invention, it is also possible to make all coatings identical, in order to get improved statistics on single measurement (i.e., for a particular binding interaction or elastic measurement).

FIG. 5 is schematic showing the use of a magnet array to measure elasticities of polymers. In this example, the bottom surface has only one coating, although it is also possible to use an array of coatings, as described for FIG. 4. If an array of coatings are used, then the bottom surface is exposed to a solution containing a number of different types of polymers and each polymer attaches exclusively to different discrete spots on the bottom grid, enabling parallel measurements.

FIG. 6 shows a magnetic array formed using a material, for example a single permanent magnet, or a single block of magnetizable or magnetic material, having an embossed surface.

FIG. 7 shows numerical calculations of the magnet field magnitudes resulting from two sets of criss-crossed current carrying wires (as shown schematically in FIG. 3) in the presence of an additional biasing magnetic field. FIG. 7a corresponds to a zoom in on the middle section of the crisscrossing wires, showing the wire grid that sets the scale for all the other parts of the Figure. In FIGS. 7b, 7c and 7d, the surfaces drawn are surfaces of constant magnetic field magnitude. The field magnitude decreases from 7b to 7c to 7d (or equivalently, the field magnitude shown is the same, but the current in the wires is decreased), demonstrating a localizing force that will hold magnetically-labeled entities inside of individual ‘magnetic tubes’ preventing mixing between adjacent cells, while also pulling these entities towards the magnet surface as the current is increased. FIG. 7e is a density plot of magnetic field magnitude, looking directly down on the wire grid. Bright (dark) regions represent regions of high(low) magnetic field.

FIG. 8 is a 1-dimensional variant of FIG. 7. In this case one of the two wires has been turned off, yielding a set of isolated ‘magnetic stripes’ takin to the isolated ‘magnetic tubes’ of FIG. 7.

FIG. 9 lists numerical calculations of the magnetic field magnitudes resulting from the wires in FIG. 3, with one wire turned off, showing the transformation from a periodically modulated field to a uniform one in the presence or absence, respectively, of an additional biasing magnetic field. FIG. 9a represents a zoom in on the middle section of the crisscrossing wires (one wire turned off), showing the wire grid that sets the scale for parts 9b and 9c. FIG. 9b represents a surface of constant magnetic field magnitude showing the modulation in magnetic field magnitude in the presence of a bias magnetic field. FIG. 9c represents a surface of constant magnetic field magnitude showing the uniform field strength resulting in the absence of any bias field. The magnetic field is switched between 9b and 9c simply by adding or removing the bias field.

FIG. 10 is a photograph of a permanent magnet prototype device. The magnets are on the order of mm in size, but it is possible to make devices which are much smaller still.

FIG. 10a is the prototype device consisting of a grid of magnets arranged in alternating fashion as suggested in earlier figures and held together (against their mutual repulsive forces) by surrounding pieces of metal. A glass cover slide seals the top. The U.S. quarter shows actual size scale. FIG. 10b is a zoom in on the magnet region (dark splotches are dirt marks only). FIG. 10c outlines a simple experiment which can be performed to map out the magnetic fields of the device. In this example, a rubber O-ring was glued to the top glass slide to serve as a containment ring. This was then filled with a solution of 5 micron superparamagnetic beads (the same beads that would be attached to the biological entities) mixed in water. As the beads settle of out the solution they drift down in the magnetic field of the grid below and effectively map out the magnetic field, leaving the chessboard pattern as predicted. Dark regions are regions where many beads have collected. Light regions are free of all beads.

FIG. 11 shows images akin to those of FIG. 10c, except that the magnetic beads have been deposited onto regular audio cassette tape that has been periodically magnetized and the image is more highly magnified. FIG. 11a and 11b show scalability, having been recorded at different frequencies. In these figures, unlike in FIG. 10c, the beads appear as bright spots. The tape track boundaries are clearly visible as are the boundaries between each of the alternating magnetization directions in the tape. For size scale, each small white spot is an individual magnetic bead with diameter 4.5 μm. Larger spots are collections of beads.

It is to be understood that the referenced figures are illustrative only and are not essential to the enablement of the claimed invention.

### DETAILED DESCRIPTION OF THE INVENTION

The invention relates in a broad sense to the quantitative analysis of elastic forces of biological molecules and binding forces between biological molecules, as well as manipulation of biological molecules. The invention provides advantages over currently available technologies. For example, the devices or articles of the invention are simple and inexpensive to manufacture, no complex microfabrication is required, and all components required for manufacture are readily available. The methods and articles provide the ability to alternate easily between single measurements and large scale parallel measurements. Large scale parallel measurements in turn provide the acquisition of a greater number of data points, leading to better statistical certainty and faster processing. The invention in based in part on the novel application to quantitative biological analyses of particular magnetic field geometries that enable forces which are several orders of magnitude greater than those achieved using currently available optical and magnetic systems.

The invention can be further used in drug development and/or screening processes. According to the methods of the invention, candidate biological agents can be identified based on their ability to bind a specific known biological molecule. Moreover, in addition to identifying the candidate agent, the method allows the strength of interaction between the candidate agent and its cognate binding partner (e.g., cognate receptor) to be determined. In fact, a panel of agents can be identified and relatively ordered based on their binding strengths. The range of manipulations that can be performed on biological agents is related to the range of forces that can be applied. In other words, the greater the range of forces (i.e., the greater the range of magnetic field gradients), the more manipulations that can be performed. Also the manipulation kinetics can proceed more quickly given a greater force. It is therefore, advantageous to be able to produce as large a force as possible, and consequently, as large a magnetic field gradient as possible. Furthermore, being able to perform many measurements simultaneously confers additional speed and efficiency advantages. It is therefore, advantageous to be able to produce this large force (and magnetic field gradient) consistently over a large spatial area.

To this end, this invention provides for the manipulation of biological entities through the use of magnetic fields (and corresponding magnetic field gradients). As further used herein, the term biological entities and biological molecules intends to embrace both biological and chemical entities which are natural or synthetic in nature. Biological entities of interest are attached (i.e., bound or conjugated) to magnetic beads (i.e., magnetically-labeled) and then manipulated by specifically tailored magnetic fields (and corresponding magnetic field gradients) designed to give large force ranges with high precision over unlimited areas for unlimited parallel processing.

For a given magnetic material, the magnetic field and magnetic field gradients (the key physical quantity in determining the force) depend on the position from the source of the magnetic field and on the geometry of the source of the magnetic field. High gradients occur when the field varies rapidly from one point to the next in space such as occurs near a sharp edge of, near a sharp point of, or near a narrow slit or groove in a magnet. This implies that to achieve the highest forces, one needs to use the geometrically smallest magnet or smallest magnet features (e.g., only the sharp comer points of a larger magnet). Moreover, this precludes one from performing large-force manipulations over extended areas since the force is only large near the small edge or point or air-gap of the magnet, and thereby precludes parallel processing over large areas. Conversely, a homogenous large area magnet may enable forces over a larger area, but because of the smaller gradients involved, these forces are all smaller than those achievable in the localized vicinity of a smaller magnet or sharp edge. Thus, the tradeoff involved in the technology available prior to the present invention has been that a stronger force limits operational sizes to small areas, and conversely that large operation areas are limited to the use of smaller forces.

The present invention overcomes or circumvents this limitation by the novel application to biological analysis systems of particular magnetization geometries that enable large and well-defined forces to be created over an unlimited area. One important application of this novel use is the ability to perform massive parallel manipulation of biological entities. Additionally the design is easily adaptable to be compatible with present-day infrastructure in the burgeoning DNA microarray chips industry.

A high field gradient over a large area is produced using an array of alternating magnetizations, rather than a single magnet, arranged in, for example, one dimensional stripes, two dimensional lattices (e.g., square, hexagonal, etc.) or chessboard-like patterns with neighboring regions being magnetized in opposite directions. Although the figures diagram a checkerboard pattern, the invention intends to embrace this and other patterns which can be achieved using the magnetic or wire arrays described herein. Thus, both regular and irregular patterns should be possible and are intended by the invention. This grid can be made in several different ways. One way is by using a set of permanent rare-earth magnets, of which NdFeB are the strongest commercially available permanent magnets. NdFeB are also relatively inexpensive, and are ideally suited to the task. However, any hard magnet material suffices provided it is not prone to self-demagnetization and has a high enough Curie demagnetization temperature to withstand whatever temperature ranges the experiments might be run over. A second way in which the grid may be formed is by writing a permanent magnet structure onto standard audio/video magnetic tape or magnetic floppy disk, using standard or slightly modified magnetic read/write technology. A third way to form the grid is to use a, preferably single, permanent magnet with an embossed (e.g., etched) surface structure with grooves cut into the surface leaving behind a patterned surface in the form of any of the arrays considered above. This third system behaves similarly to the first system mentioned above and is physically equivalent to the first system with every second magnet missing in the grid (giving a reduction in force of a factor of two) and with an additional biasing magnetic field applied. As used in the specification, and unless otherwise stated, “permanent magnet” refers to all of the magnets mentioned above. A fifth way of making the grids consists of using a magnetizable material with the same geometry as the permanent magnet material used in the third method of constructing the grid described above or with any regular grid structure. This magnetizable material is then placed in an externally applied uniform magnetic field which magnetizes it and results in a system physically similar to the third method described above, except for the additional uniform magnetic field present. A sixth way in which a grid may be formed is by using a series of “criss-crossing” wires through which flowing electric currents generate similar magnetic fields (and corresponding magnetic field gradients). A fourth way of making the grid involves a combination of any of the above.

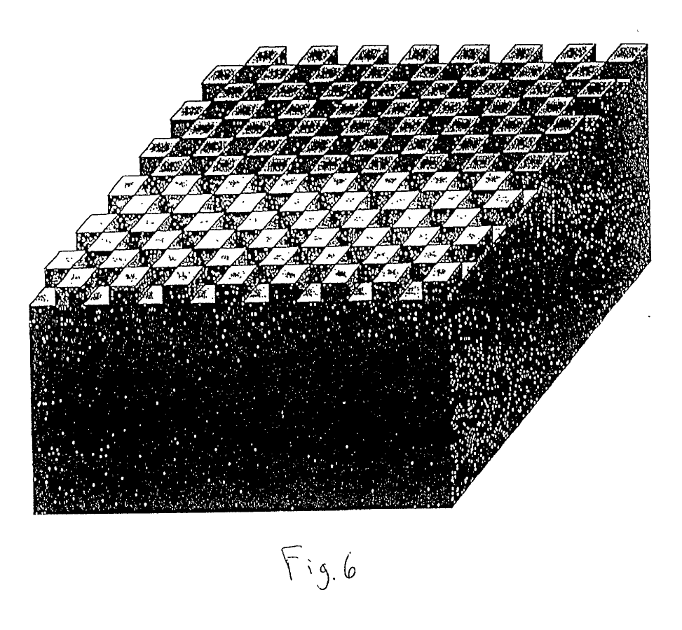

One and two dimensional magnetic arrays can also be formed using a single permanent magnet with a surface which has been embossed (e.g., etched or carved) to give an array structure on the surface as shown, for example, in FIG. 6. It should be understood that any embossing pattern may be utilized. When looked at in cross section, this magnet looks effectively like a surface with many regularly spaced bumps on it. In effect, this is physically equivalent to the permanent magnet array but with every second magnet missing. The fields produced are similar to those produced using an arrangement of alternating permanent magnets (as described above) but smaller by a factor of two. Another way of forming one and two dimensional arrays is by using magnetizable material which either has an embossed surface (as described above), or is simply itself a grid (e.g., similar to a wire insect netting or mesh). When this latter type of arrangement is placed into an externally applied, preferably uniform, magnetic field, it will magnetize and behave just as the above embossed magnet behaves. It is important to note that the last two embodiments provide a modulated field.

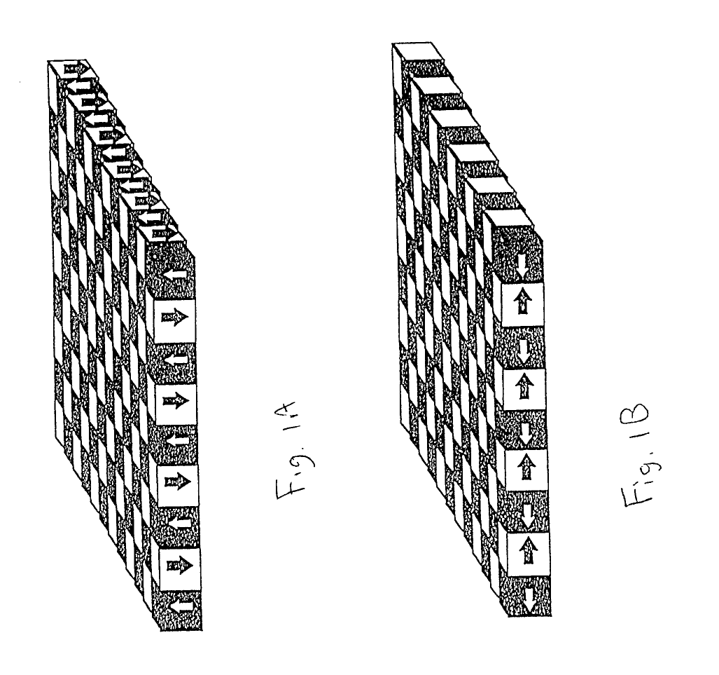

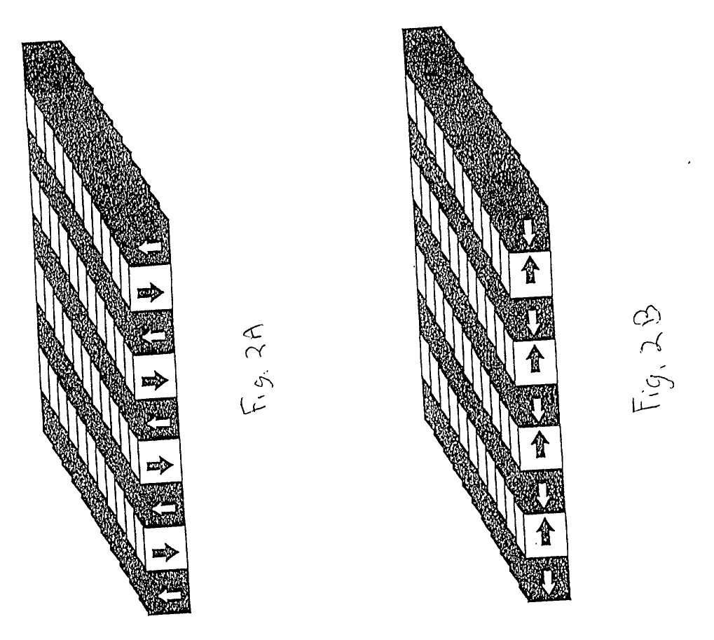

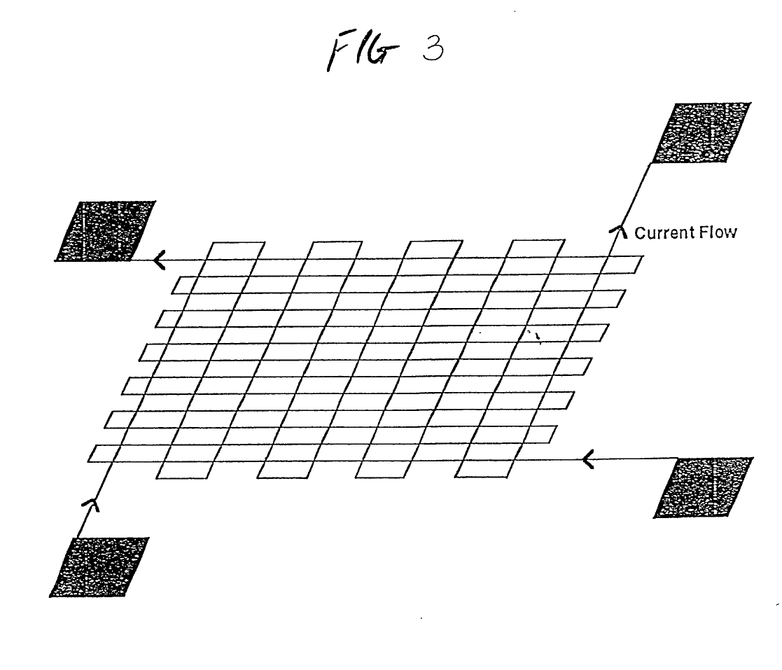

A high field gradient (essential for creating a high magnetic force) is produced in the perpendicular direction towards the planar 2-D array of magnets. Close to the surface of the magnet array, the field is dominated by just the single small magnet in that area, while further from the surface, the fields of neighboring magnets rapidly cancel each other. The rapid cancellation with distance from the surface gives a large gradient and force everywhere above the array, with the force being in the direction perpendicular to the plane of the array, and with the force being well-defined everywhere. Examples of some of these possible arrays include but are not limited to those shown schematically in FIGS. 1, 2 and 3. The z-direction is oriented perpendicular to the plane and is the direction in which the primary force acts. In any parallel x-y plane the magnetic field direction produced by the array rotates in accordance with the alternating directions of the adjacent magnets comprising the magnet array. The rotation in space of the field vector direction enables two distinct embodiments of the invention described here.

Magnetic or magnetizable particles which can include paramagnetic, superparamagnetic, or ferromagnetic particles, and which are assumed to be spherical only for simplification in the present application (but which can be non-spherical as well as other shapes), will rotate their magnetic moments to always align themselves with the magnetic field direction from the array of magnets. Because of this, it is only the change in the field magnitude (and not the change in the field direction) that determines the force acting on the magnetically-labeled biological entities. This creates an approximately uniform force over any parallel plane over the grid of magnets (i.e., independent of x, y position). This gives a method to pull, in the z-direction, on biological entities with a large approximately constant force over an unlimited area. As used herein, “approximately uniform” intends a variation within 20%. In preferred embodiments, the variation is within 10% and in even more preferred embodiments, the variation is within 5%. The degree of uniformity can essentially be made as high as required. Methods for dealing with residual variation in field magnitude have been identified and described in Zabow et. al, “Improving the Specularity of Magnetic Mirrors For Atoms”, Eur. Phys. J. D. 7, 351-359 (1999), the entire contents of which is incorporated herein by reference.

In an alternate embodiment of this invention, it is possible to create a spatially periodic modulation of the force with periodicity determined by the periodicity of the grid of magnets. This can be accomplished by simply placing the grid in an externally applied offset (i.e., biasing) magnetic field, or can be made to occur automatically depending on the choice of array structure used. The resultant magnetic field is then composed of the spatially rotating field of the array of magnets plus the field of the single large-area magnet which always points in one direction. When the rotating magnet field points in the same direction as the bias field, the two fields add; when it points anti-parallel, the two fields subtract giving a periodic modulation in magnetic field magnitude. This create “lines” (i.e., one dimensional) or “tubes” (2 dimensional) of magnetic field maxima in grid patterns (e.g., chessboard-like patterns) extending out from the array of magnets. This implementation therefore has both a pulling force in the z-direction as well as an additional confining force in the x-y plane, which would prevent unwanted mixing of different biological entities as they are attracted towards the magnet array, or which could be used in elasticity experiments to exert well-defined transverse forces in addition to the primary longitudinal stress forces simply by varying the biasing field or moving the array appropriately.

A solution of magnetically-labeled biological molecules (e.g., magnetic beads attached (i.e., covalently bound or conjugated) to a biological molecule) is deposited onto a surface which is pre-coated uniformly with a biological coating or with a 1-dimensional or a 2-dimensional array of spots each containing a different biological molecule much like the presently very popular gene micro- and macro-arrays. The magnetically-labeled biological agents then (1) settle down onto the surface because of gravity, or (2) can be accelerated towards the surface using an additional magnet as in a magnetic separator, or (3) contact the bottom (i.e., first) surface through natural diffusion in the liquid. Depending on the biological molecule and the coating on the bottom surface, the magnetically-labeled biological molecule will adhere (i.e., bind or attach) with different strengths at different locations. If the bottom (i.e., first) surface is uniformly coated with the same coating, then the biological agents will bind with the same strength throughout the bottom surface.

As described herein, the coating on the first surface can be a wide variety of components including a binding partner (i.e., a binding member) of the biological molecule (e.g., the cognate receptor of the binding partner). For example, the coating can be a solution of an extracellular matrix protein such as collagen or fibronectin to which a magnetically-labeled biological molecule such as an integrin (e.g., fibronectin receptor) may be bound. In this latter example, the binding interaction (i.e., binding strength) between the extracellular protein and the integrin can be measured by applying the magnetic field via the second surface, as described below. The coating may alternatively be an inert composition which is used solely to secure the biological molecule (e.g., the binding partner or a polymer) to the first surface. This is the more likely scenario when the intention is to measure an elastic strength of a biological molecule such as a polymer. In this latter instance, the polymer is prepared with a free end which can interact and ultimately bind to the coated surface, and with the opposite end modified such that it is bound to a magnetic bead. As an example of this embodiment, a polymer such as DNA can be bound to a bottom surface coated with polylysine which avidly binds nucleic acids. In preferred embodiments, the strength of polymer binding to the coating is greater than the elastic strength within the polymer, since this ensures that the polymer will not be pull away from the bottom surface after the magnetic field is applied. In still other embodiments, the polymer or binding member (or receptor, depending on the configuration) can be synthesized directly on the first surface using for example solid phase synthesis techniques.

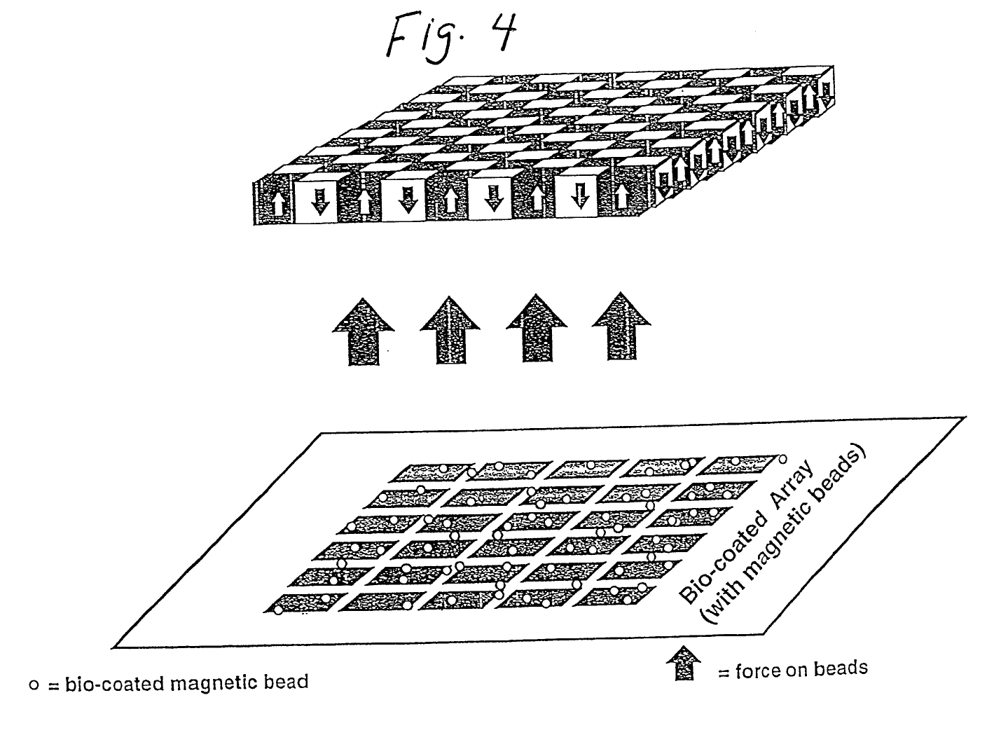

After the bottom (i.e., first) surface is coated and contacted with the magnetically-labeled biological molecule, the magnet array of the opposite surface (e.g., the second surface) is then brought in, for example, from above, as shown in FIG. 4. It is important to note, however, that generally the magnetic field strength completely overwhelms any gravity forces, so that the direction of approach may be any convenient direction for the user. Thus although the discussions containing herein refer to the vertical direction, this is only one example, and is not intended to be limiting. For a permanent magnet array, the force it produces pulling upwards on the magnetically-labeled biological molecules on the bottom surface can be controlled by moving the array closer to, or further from, the bottom surface. If the array is composed of criss-crossing wires (as is the case with electric current carrying wires), then no mechanically moving parts are required and the forces can be controlled simply by adjusting the currents flowing in the wires. Alternatively, for an array of magnetizable material placed in an externally applied magnetizing field, the array may either be mechanically moved or the external field applied may be varied and the array held fixed. Both methods may be useful dependent upon the situation. It is possible then by observing the magnetic particles bound to the receptor to determine the binding forces acting between the binding members and their receptors at the various sites of the “bio-array”. As mentioned earlier, the first surface can be either uniformly coated with a single species or with a plurality of species. A plurality of species is useful if the aim is to measure simultaneously a number of different interactions. If the first surface is coated with a single species, then it is still possible to measure simultaneously a number of different interactions, provided that a plurality of magnetically-labeled biological molecules of different species are separately applied to discrete regions of the first surface. It may be preferable, if multiple simultaneous measurements of different interactions are desired, to vary the coating (which includes the binding member) applied to the discrete regions of the first surface, and then apply a uniform solution or suspension of one species of magnetically-labeled biological molecules (including cognate or putative cognate receptors).

It is to be understood that the weakest strengths will be those to be measured by the invention, namely the binding strength between the binding member and a receptor or an elastic strength of a molecule. In other words, the binding strength between the binding member, receptor or molecule attached or conjugated or bound to the first surface will always be greater than the binding strength between a binding member and receptor or an elastic strength within a molecule. In addition, the binding strength between the magnetic particle and the biological molecule to which it is conjugated (e.g., a binding member or a receptor depending on the configuration of the system, or biological molecule such as a polymer), will always be greater than the binding strength between a binding member and a receptor or an elastic strength in a molecule.

As used herein, the magnetic force relates to the product of the magnetic field gradient and the magnetization capability of the magnetic beads (e.g., a fixed magnetization as for saturated ferromagnetic particles, or a magnetization functionally dependent on the applied magnetic field magnitude as for paramagnetic or superparamagnetic particles) which are bound to the biological molecule. Thus, the magnetic force is dependent upon the type of magnetic material used. The magnetic force experienced by the binding member/cognate receptor interaction is a function of the distance of the top magnetic array from the bottom surface (for the case of permanent magnets) or of the current flowing in the crisscrossing grid of wires, or of the externally applied magnetic field in the case of a grid of magnetizable material, all of which can easily be measured.

Determining whether a magnetic particle attached to the biological molecule (such as for example, the receptor), and concurrently the magnetically-labeled biological molecule, has remained attached to any spot on the bottom surface, because the binding strength between the binding member and the cognate receptor is greater than the magnetic force applied, can be accomplished in several simple ways. A standard optical microscope can be used to examine the bottom surface. Although the magnetic particles may be small even on the scale of the wavelength of light, they still scatter sufficient light to be easily seen with a standard microscope.

Alternatively, as may be useful if there are vastly different light scattering properties in each spot on the bottom surface (due to different optical properties of the different biological coatings), it is possible to place a thin piece of a removable adhesive (e.g., adhesive tape) directly onto the underside surface of the top surface (i.e., the magnetic array). Magnetic beads removed from the bottom surface will be pulled up and, for appropriately prepared tape, will adhere to the removable tape. This tape is spatially uniform and so avoids possible spurious results due to variations in light scattering properties of the different spots on the bottom array. The piece of tape, either left attached to the top magnet, or removed, can then itself be examined in several ways. One way involves the use of an optical microscope through standard light-scattering. Another way to detect the magnetic particles on the adhesive surface is to use fluorescent beads. In this way, spots on the tape at which beads are attached can be made to fluoresce, by, for example, sandwiching an optical waveguide between the tape and magnet array and using the exponentially decaying light field leaking out the guide to make only the beads stuck directly to the tape (i.e., sufficiently near to the optical wavelength) fluoresce. Yet another way to detect magnetic beads on the adhesive is to feed the adhesive tape through some suitable form of automated magnetic reader, for example, a reader akin to a magnetic audio tape or disc drive read head. Such automated magnetic readout systems are particularly useful for detecting magnetic beads at high feed rates. Such systems may even become vital as densities of elements in the arrays increase or as smaller more tightly packed beads no longer provide distinguishable light scattering centers.

It is clear therefore, that the ability to pull the beads up onto some other surface (which may be more freely chosen for convenience of further operation) becomes important. If the magnetic particles accumulating on the top surface are examined, it is important to ensure that particles being pulled off some particular spot on the bottom surface, are pulled straight up so that there is no mixing between particles pulled off adjacent spots. Such mixing would effectively erase the information on the difference in binding forces between the coated magnetic particles and the different biological coatings at different points on the bottom surface and would foil the parallel nature of the analysis being performed. Although the magnetic force exerted is directly upwards, the amount of inherent diffusion in the liquid will determine how far the particles randomly stray to one or other side. If there is little straying, then the first implementation of magnetic array suffices; for larger perturbing sideways motions, the second implementation (with the bias field) can be used to provide confining tubes up which the particles travel, thereby preventing any mixing. Additionally, for the case of small cell sizes in the grid, which necessitate bringing the two surfaces close together, perhaps even into physical contact with each other, the confining magnetic tubes will be useful to reduce sideways motions of the beads (or particles) as may occur from resulting capillary forces, etc.

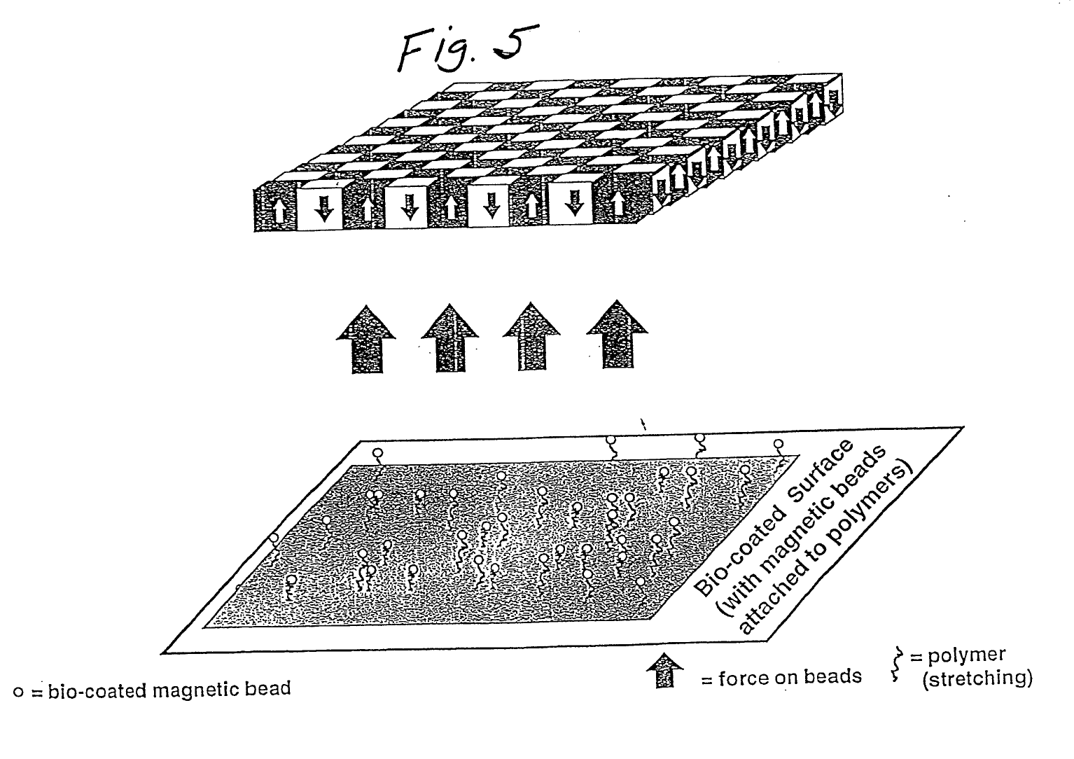

The methods and apparatus of the invention also allow parallel measurements of the elastic strengths of polymers. If the bottom surface is coated such that one end of a long polymer sticks to it (as described above), while the other end of the polymer sticks to a suitably coated magnetic bead, the same forces described above can be used to pull upwards on the magnetic beads thereby stretching the polymers (see FIG. 5). Observation of the heights of the beads in the solution as a function of the force (either determined by the position of permanent magnet array from the bottom surface or the amount of electric current flowing through wire grid or the size of the externally applied magnetic field in the case of a grid of magnetizable material) gives information relating to the elasticity or elastic strength of the polymers. For the case of the implementation with periodic force modulation in space over any x-y plane, suitable sideways motion of the magnet array or suitable variation of the direction of the bias magnetic field applied (to shift the spatial location of constructive and destructive magnetic field superpositions) may also yield useful off-axis information.

Since the geometries of the magnetic arrays described above are essentially identical to that of the rapidly increasing number of gene-chip or DNA microarrays becoming commercially available, the systems and methods described herein can be easily incorporated into many biological analyses including diagnostic procedures. Additionally, the system is simple and inexpensive to fabricate. The array is inexpensive to manufacture, since at a minimum it is a simple checkerboard grid of small magnets or magnetizable material, or a magnetic structure written on a magnetic recording media (such as tape or disk), or an array of wires easily micro-fabricated using standard lithography techniques. The magnetic beads are readily available from an already established industry in magnetic separation technology, with commercial suppliers such as, but not limited to, Dynal, Boehringer-Mannheim and Promega. Moreover, it is already standard for these beads to be purchased with a wide variety of biological coatings already attached (e.g., from Dynal). Finally, the bottom surface containing a grid of different biological entities (i.e., coating and binding members) can be purchased from any one of numerous companies now producing such DNA- chips and now offering also custom chips.

The system provided by the invention is particularly well suited to miniaturization. For the case of a magnetic array of a set of permanent magnets, off-the-shelf magnets are of a suitable size to yield comparable grid periodicities to that offered by available DNA macroarrays. For tape/disk written magnetic arrays, sizes can easily be made comparable to, or even smaller than available DNA microarrays. For current-carrying wire implementations or etched (e.g., embossed) surface structures, standard lithography can produce grids of sizes comparable to the most densely packed microarrays available today as well as grids of considerably smaller size too, in anticipation of the likely miniaturization of present-day gene-chip sizes.

The physics underlying the large forces produced by these arrays also lends itself well to miniaturization. The force strength produced by this magnetic array is proportional to the product of magnetic particle magnetization and k*exp (−kz), where k is a constant inversely proportional to the spacing between magnets or wires. Therefore, as the size of each element in the array shrinks, not only can the number of elements vastly increase, but also the maximum forces, giving a greater force range to work with. Additionally, from a simple geometric viewpoint, shrinking sizes necessarily lead to shorter processing times. These points, together with the magnetic array's geometric resemblance to DNA-chips, has lead to the term “microarrays” when describing these magnetic grids.

As a convenient benchmark for typical forces required, it should be noted that one of the stronger biological bonds is that of biotin-avidin of around 100 pN. Initial experiments and extrapolating calculations show that such forces and indeed even forces orders of magnitude stronger are readily achievable with the magnetic arrays described here. For example, a force on the order of 100 nN has been achieved using a 20 micron spacing grid and using Dynal magnetic beads (4.5 micron diameter). Though it is to be understood that larger than 100 nN forces may be produced by using one or more of the following: larger diameter beads, stronger magnetic beads, and smaller grid patterns. This experimental work is in contrast to typical forces of no greater than order 10 pN available with the technology of optical tweezers which has often been applied to biological studies. Not only are the forces of the magnetic array several orders of magnitude greater than that of optical tweezers and at least an order of magnitude greater than available magnetic tweezers, but the force is the same over an unlimited area as opposed to a single spot (e.g., the laser focus in the case of optical tweezers, or the single sharp edge of a single magnet in previous magnetic devices).

The invention provides methods for the measurement of elastic strengths of biological molecules and for the measurement of binding strengths between biological molecules. As used herein, a binding strength is measured between two molecules. For the sake of clarity, one molecule of the pair is referred to as a binding member or binding partner, and the other is referred to as a cognate receptor. A binding member and a cognate receptor refer to two molecules which are capable of specific physical interaction between each other (i.e., binding to each other). In effect, either molecule of the pair can be the binding partner or the receptor. As used herein, the term receptor includes but is not limited to molecules which are commonly referred to as receptors in the art such as for example cell surface receptors, growth factor receptors and hormone receptors. Binding members and receptors may be selected from a wide range of biological and chemical molecules including but not limited to nucleic acids such as DNA and RNA; peptides and polypeptides such as cell surface receptors (and their ligands), integrins, extracellular matrix proteins, plasma proteins, cell signaling proteins including signaling adaptors, kinases, and phosphorylases, antibodies, transcription factors, DNA binding proteins, and RNA binding proteins; carbohydrates and lectins and lipids. The methods of the invention can be used to identify previously unknown binding partners of a biological or chemical molecule, or to identify agonists or antagonists of a biological or chemical molecule, or to determine the binding strength of one or more binding member/cognate receptor interactions. As used herein, either a binding member or a receptor may be bound to either the first surface or the second surface. Therefore, the invention intends to embrace configurations in which the binding member is magnetically-labeled and the receptor which is then bound to the first surface, is not magnetically-labeled. The decision of which molecule (i.e., the binding member or the receptor) to label magnetically will depend upon the particular analysis being conducted and the facility with which either can be labeled. Making this determination is well within the realm of the ordinary artisan.

The methods can also be used to measure elastic strengths of biological molecules. An elastic strength as used herein is a measure of the extent to which a biological molecule may be stretched. In one embodiment, the extent to which the biological molecule may be stretched without undergoing an irreversible change in its structure (e.g., a breaking of bonds such as amino bonds in a peptide or phosphodiester bonds in a nucleic acid) may be of interest. In still another embodiment, the amount of force required to “break” or sever a biological molecule into two separate entities (e.g., a nucleic acid which has been severed into two pieces) may be of interest. Both force measurements are readily available using according to the methods of the invention. Examples of biological molecules which can be analyzed in this manner include nucleic acids; peptides; synthetic polymers; and cytoskeletal proteins such as for example actin. In preferred embodiments, the elastic strength of biological molecules such as polymers is to be determined. A polymer is broadly defined as a macromolecule made up of repeating monomer units. Preferred polymers include those biological molecules listed above.

The biological molecules to be tested for either elastic or binding strength are preferably labeled with a magnetic particle (i.e., magnetically-labeled). Many varieties of magnetic particles are currently available including paramagnetic, ferromagnetic and superparamagnetic particles. In a preferred embodiment, the particles are made from superparamagnetic material (e.g., because easy demagnetization facilitates easier recycling of beads). However, for maximizing forces, the use of ferromagnetic particles may be alternatively desired. Superparamagnetic materials are materials which, in the absence of a magnetic field, are not magnetic but which, in the presence of a magnetic field, become highly magnetic. A discussion of further magnetic materials can be found in U.S. Pat. No. 5,411,863, the entire contents of which are incorporated by reference herein. When a biological molecule such as a polymer is to be labeled with a magnetic particle, it is preferred that only one end of the polymer is labeled. In this way, the other end of the polymer is free to bind a surface, such as the first surface in the devices of the invention. When the biological molecule to be labeled is for example a binding partner or a receptor and if a binding site is known, then preferably the magnetic particle is conjugated elsewhere on the molecule so as not to interfere with binding.

Yet another application of the systems and apparatus described herein is in the field of drug discovery. For example, libraries can be generated of compounds (i.e., library members) with a magnetic particle at a fixed position. Alternatively, libraries of compounds can be used as the “coating” to the first surface. By identifying a subset of library members that are capable of binding to a biological molecule (e.g., a known receptor or polypeptide), it is then possible with further screening to isolate individual library members that bind to the biological molecule. In this way, a spectrum of compounds can be identified with increasing or decreasing binding strengths to a particular biological molecule.

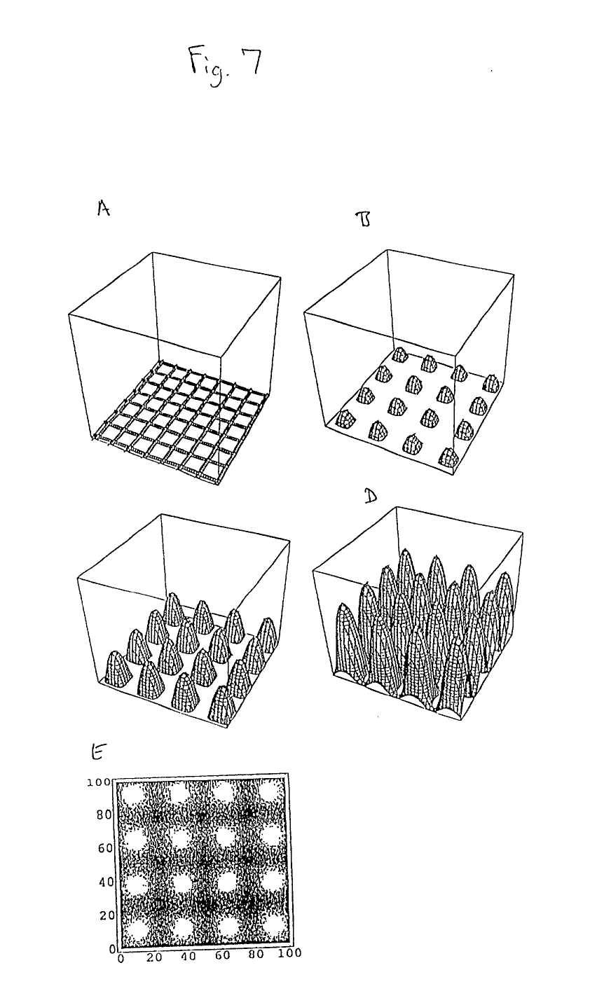

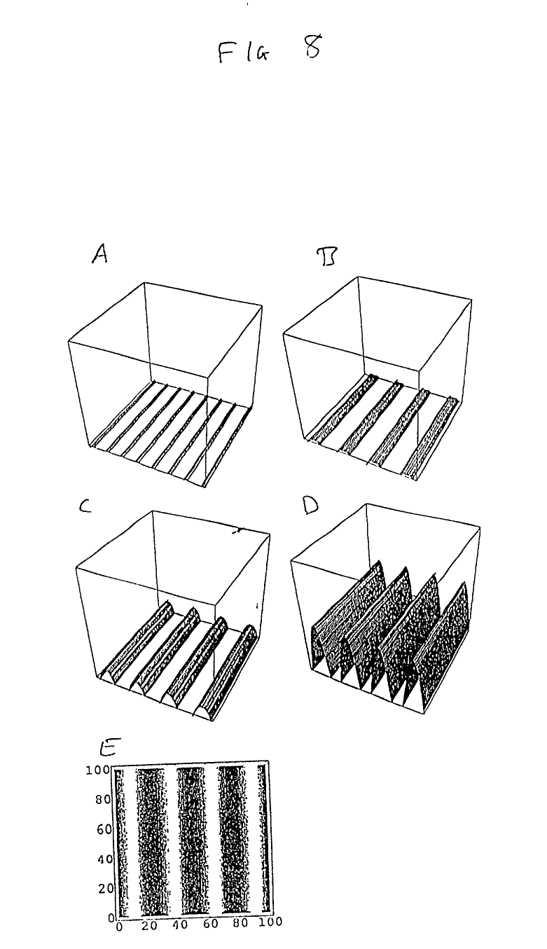

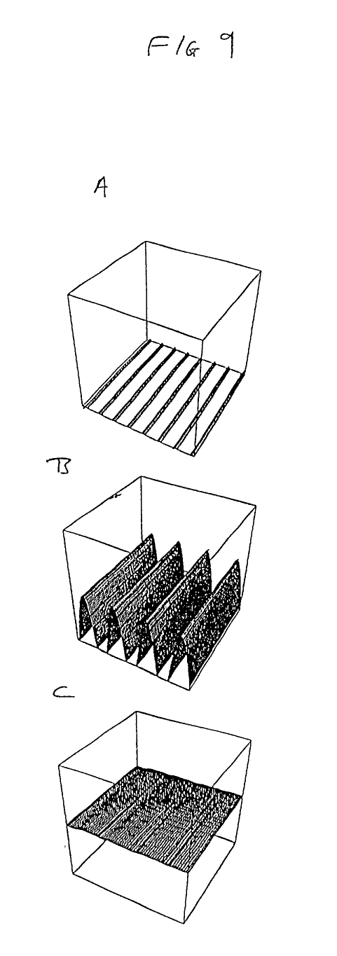

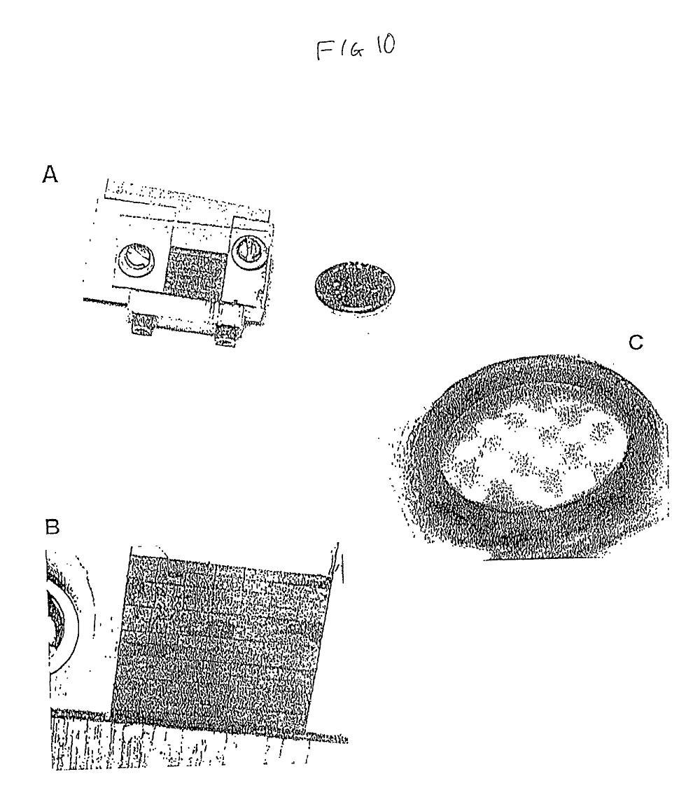

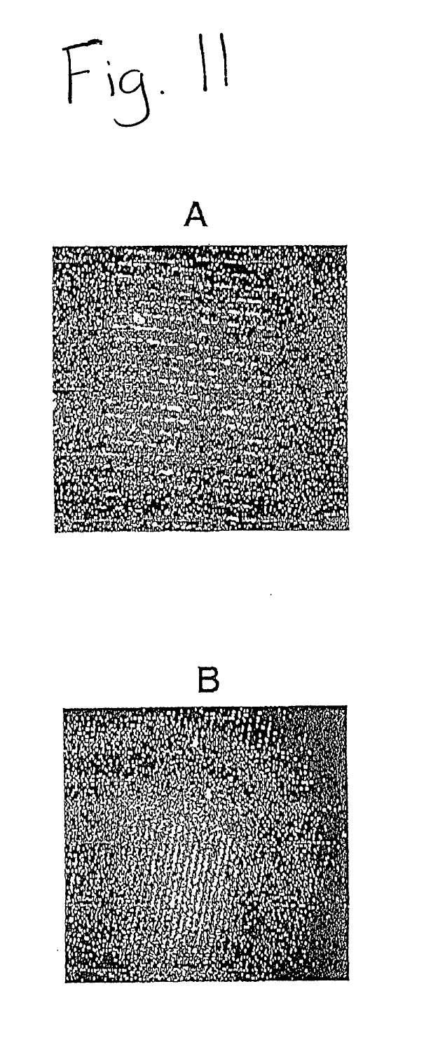

In another application, the methods and apparatus described herein may be used to promote mixing of magnetically-labeled biological molecules, for example, in microfluidic systems. In some microfluidic systems, separate fluid streams may be used to transport different types of biological molecules. The separate streams may be combined to form a single stream which transports the different types of biological molecules to a location at which a desired operation (e.g., analysis) is performed. Because of the tendency of the separate streams to flow laminarly within the system, different types of biological molecules may be segregated even after the separate streams are combined to form a single stream. The methods and apparatus of the invention can be used to generate magnetic fields that promote mixing of the different biological molecules within the single stream. In these embodiments, the microfluidic system can include a magnetic grid as a substrate over which the stream flows. The magnetic fields generated by the grid can manipulate the magnetically-labeled biological molecules within the streams, thereby promoting mixing of the different types of molecules. Any of the magnetic grids described herein may be used. For example, the grid shown in FIG. 7 may be used to provide magnetic fields extending upward from the grid surface (as shown in FIGS. 7B to 7D) into the flowpath of the molecules. In some embodiments, the magnetic fields may be shifted laterally in space to provide additional mixing, for example, by varying one or more of the magnitude, the direction, or the position of the biasing field. The mixing provided by the systems and apparatus of the invention can result in a relatively uniform distribution of the different types of biological molecules throughout a single stream which may be important in certain microfluidic systems.

### Equivalents

Those skilled in the art will recognize, or be able to ascertain using no more than routine experimentation, many equivalents to the specific embodiments of the invention described herein. Such equivalents are intended to be encompassed by the following claims.

All references, patents and patent applications disclosed herein are incorporated by reference in their entirety.

We claim:

1. A method for measuring a binding strength between at least one binding member and a cognate receptor comprising
contacting a magnetically-labeled cognate receptor with an at least one binding member conjugated to a first surface,
applying a magnetic force to the magnetically-labeled cognate receptor by positioning the first surface in proximity to a second surface having a magnetic field and magnetic field gradient, and
determining whether the magnetically-labeled cognate receptor remains in contact with the at least one binding member,
wherein the magnetic field and the magnetic field gradient are produced by an arrangement selected from the group consisting of an antiparallel adjacent array of permanent magnets, an antiparallel array of electric current carrying wires, a magnetic media with a one- or two-dimensional periodic pattern, a permanent magnet with a regularly embossed one or two dimensional surface structure, and a magnetizable material formed in a one or two dimensional grid, or with an embossed one or two dimensional surface structure in the presence of an externally applied uniform magnetic field.

2. The method of claim 1, wherein the first surface has an area of at least 1 mm.

3. The method of claim 1, wherein the magnetic field is approximately uniform throughout an area of the second surface.

4. The method of claim 1, further comprising varying the magnetic field.

5. The method of claim 4, wherein the magnetic field is varied by changing a distance between the first surface and the second surface.

6. The method of claim 4, wherein the magnetic field is varied by changing an electric current.

7. The method of claim 1, wherein the at least one binding member is at least 100 binding members.

8. The method of claim 1, wherein the at least one binding member is at least 10,000 binding members.

9. The method of claim 1, wherein the first surface is comprised of a plurality of discrete regions.

10. The method of claim 9, wherein a different species of the at least one binding member is conjugated to each of the plurality of discrete regions.

11. The method of claim 1, wherein the first surface is at least 1 μm from the second surface.

12. The method of claim 1, wherein the first surface is a microscope slide.

13. The method of claim 1, wherein the magnetic field gradient is at least 0.1 Tesla/mm.

14. The method of claim 4, wherein the magnetic field is varied by changing the externally applied uniform magnetic field.

15. A method for measuring an elastic strength of a biological molecule comprising
binding a biological molecule which is labeled with a magnetic particle at one end to a first surface,
applying a magnetic force to the biological molecule by positioning the first surface in proximity to a second surface having a magnetic field and a magnetic field gradient, and
determining a distance between the first surface and the magnetic particle as a measure of the elastic strength of the biological molecule after the magnetic force has been applied,
wherein the magnetic field and the magnetic field gradient are produced by an arrangement selected from the group consisting of an antiparallel adjacent array of permanent magnets, an antiparallel array of electric current carrying wires, a magnetic media with a one- or two-dimensional periodic pattern, a permanent magnet with regularly embossed one or two dimensional surface structure, and a magnetizable material formed in a one or two dimensional grid, or with an embossed one or two dimensional surface structure in the presence of an externally applied uniform magnetic field.

16. The method of claim 15, wherein the first surface has an area of at least 1 mm².

17. The method of claim 15, wherein the magnetic field is approximately uniform throughout an area of the second surface.

18. The method of claim 15, further comprising varying the magnetic field.

19. The method of claim 18, wherein the magnetic field is varied by changing a distance between the first surface and the second surface.

20. The method of claim 18, wherein the magnetic field is varied by changing an electric current.

21. The method of claim 15, wherein the biological molecule is at least 100 biological molecules.

22. The method of claim 15, wherein the biological molecule is at least 10,000 biological molecules.

23. The method of claim 15, wherein the first surface is comprised of a plurality of discrete regions.

24. The method of claim 23, wherein a different species of biological molecule is conjugated to each of the plurality of discrete regions.

25. The method of claim 15, wherein the first surface is at least 1 μm from the second surface.

26. The method of claim 15, wherein the first surface is a microscope slide.

27. The method of claim 15, wherein the magnetic field gradient is at least 0.1 Tesla/mm.

28. The method of claim 18, wherein the magnetic field is varied by changing the externally applied uniform magnetic field.

29. A method for measuring a binding strength between at least one binding member and a cognate receptor comprising
contacting a magnetically-labeled cognate receptor with an at least one binding member conjugated to a first surface,
applying a magnetic force to the magnetically-labeled cognate receptor by positioning the first surface in proximity to a second surface having a magnetic field and a magnetic field gradient, and
determining whether the magnetically-labeled cognate receptor remains in contact with the at least one binding member,
wherein the second surface has an area of at least 1 mm², and a magnetic field magnitude produced is approximately uniform throughout planes parallel to the first surface and the second surface.

30. The method of claim 29, further comprising varying the magnetic field of the second surface.

31. The method of claim 29, wherein the magnetic field is produced with an adjacent array of antiparallel permanent magnets, or with magnetic recording media comprising an array of anti-parallel recorded magnetizations, or with a permanent magnet with an embossed surface structure in the form of a one or two dimensional grid.

32. The method of claim 31, wherein the magnetic field is varied by changing a distance between the first surface and the second surface.

33. The method of claim 29, wherein the magnetic field is produced with an antiparallel array of electric current carrying wires.

34. The method of claim 33, wherein the magnetic field is varied by a change in electric current.

35. The method of claim 29, wherein the second surface has an area of at least 1 cm².

36. The method of claim 29, wherein the magnetic field gradient is at least 0.1 Tesla/mm.

37. The method of claim 29, wherein the first surface is comprised of a plurality of discrete areas.

38. A method for measuring an elastic strength of a biological molecule comprising
binding a biological molecule which is labeled at one end with a magnetic particle to a first surface,
applying a magnetic force to the biological molecule by positioning the first surface in proximity to a second surface having a magnetic field and a magnetic field gradient, and
determining a distance between the first surface and the magnetic particle as a measure of the elastic strength of the biological molecule after the magnetic force has been applied,
wherein the second surface has an area of at least 1 mm², and a magnetic field magnitude produced is approximately uniform throughout planes parallel to the first surface and the second surface.

39. The method of claim 38, further comprising varying the magnetic field of the second surface.

40. The method of claim 38, wherein the magnetic field is produced with an adjacent array of antiparallel permanent magnets, or with magnetic recording media comprising an array of anti-parallel recorded magnetizations, or with a permanent magnet with an embossed surface structure in the form of a one or two dimensional grid.

41. The method of claim 40, wherein the magnetic field is varied by changing a distance between the first surface and the second surface.

42. The method of claim 38, wherein the magnetic field is produced with an antiparallel array of electric current carrying wires.

43. The method of claim 42, wherein the magnetic field is varied by a change in electric current.

44. The method of claim 38, wherein the second surface has an area of at least 1 cm².

45. The method of claim 38, wherein the magnetic field gradient is at least 0.1 Tesla/mm.

46. The method of claim 38, wherein the first surface is comprised of a plurality of discrete regions.

47. A method of mixing magnetically-labeled biological molecules comprising:
transporting a first type and a second type of magnetically-labeled biological molecule in a fluid stream, and
mixing the first type and a second type of magnetically-labeled biological molecule within the fluid stream by applying a magnetic force using an arrangement selected from the group consisting of an antiparallel adjacent array of permanent magnets, an antiparallel array of electric current carrying wires, a magnetic media with a one- or two-dimensional periodic pattern, a permanent magnet with a regularly embossed one or two dimensional surface structure, and a magnetizable material formed in a one or two dimensional grid, or with an embossed one or two dimensional surface structure in the presence of an externally applied uniform magnetic field.

48. The method of claim 47, comprising transporting the first type and the second type of magnetically-labeled biological molecule in a microfluidic system.

49. The method of claim 47, wherein more than two types of species.

50. An apparatus for measuring a biological parameter comprising
a first surface comprised of a magnetic field and a magnetic field gradient generated by an arrangement selected from the group consisting of a planar, adjacent, antiparallel array of a plurality of permanent magnets, a planar, adjacent antiparallel array of magnetizations recorded onto a magnetic recording media, a permanent magnet with regularly embossed one or two dimensional surface structure, and a magnetizable material formed in a one or two dimensional grid, or with an embossed one or two dimensional surface structure in the presence of an externally applied uniform magnetic field, and
a second surface,
wherein the magnetic field and magnetic field gradient is periodically modulated over an area of the first surface and decays non-linearly with distance from the first surface,
wherein each of the plurality of permanent magnets, magnetizations or embossed surface structure has an area of less than 1 cm².

51. The apparatus of claim 50, wherein the planar, adjacent, antiparallel array is a linear array and the first surface has an area of at least 2 cm².

52. The apparatus of claim 50, wherein the plurality of permanent magnets, magnetizations, or embossed surface structures is at least 10.

53. The apparatus of claim 50, wherein the planar, adjacent, antiparallel array is a two dimensional array.

54. The apparatus of claim 53, wherein the plurality of permanent magnets, magnetizations, or embossed surface structures is at least 100.

55. The apparatus of claim 53, wherein the plurality of permanent magnets, magnetizations, or embossed surface structures is at least 1000.

56. The apparatus of claim 50, wherein the area of the first surface is at least 1 mm².

57. The apparatus of claim 50, wherein the area of the first surface is at least 1 cm².

58. The apparatus of claim 50, wherein the area of the first surface is at least 100 cm².

59. The apparatus of claim 50, further comprising an adhesive material contacting the first surface.

60. The apparatus of claim 50, wherein the biological parameter is a binding strength between a binding member and its cognate receptor.

61. The apparatus of claim 50, wherein the biological parameter is an elastic strength of a biological molecule.

62. The apparatus of claim 51, wherein the first surface has an area of less than 1000 cm².

63. The apparatus of claim 50, wherein the planar, adjacent, antiparallel array is a linear array and further comprising a second surface.

64. The apparatus of claim 50, wherein the planar, adjacent, antiparallel array is a linear array and further comprising a magnetic particle in contact with the first surface.

65. The apparatus of claim 50, wherein the planar, adjacent, antiparallel array is a linear array and wherein the plurality of permanent magnets, magnetizations, or embossed surface structures is at least 50.

66. An apparatus for measuring a biological parameter comprising
a first surface comprised of a magnetic field and a magnetic field gradient generated by a planar, adjacent, antiparallel array of a plurality of permanent magnets, or a planar, adjacent antiparallel array of magnetizations recorded onto a magnetic recording media,
wherein the magnetic field is approximately uniform over an area of the first surface and decays non-linearly with distance from the first surface,
wherein each of the plurality of permanent magnets or magnetizations has an area of less than 1 cm², and wherein the first surface has an area of at least 2 cm².

67. The apparatus of claim 66, wherein the planar, adjacent, antiparallel array is a linear array.

68. The apparatus of claim 67, wherein the plurality of permanent magnets or magnetizations is at least 50.

69. The apparatus of claim 66, wherein the first surface has an area of less than 1000 cm².

70. The apparatus of claim 67, further comprising a second surface.

71. The apparatus of claim 67, further comprising a magnetic particle in contact with the first surface.

72. An apparatus for measuring a biological parameter comprising
a first surface with a magnetic field and a magnetic field gradient comprised of a first planar, antiparallel array of electric current carrying wires,
wherein the magnetic field is a periodically modulated throughout an area of the first surface and decays non-linearly with distance from the first surface, and wherein the first surface has an area of at least 2 cm².

73. The apparatus of claim 72, further comprising a second planar, antiparallel array of electric current carrying wires overlaid on and perpendicular to the first planar, antiparallel array.

74. The apparatus of claim 72, wherein the area of the first surface is at least 100 cm².

75. The apparatus of claim 72, further comprising a second surface having a plurality of a discrete regions.

76. The apparatus of claim 75, wherein the first surface and the second surface are separated by at least 1 μm.

77. The apparatus of claim 75, wherein the second surface is conjugated to a biological molecule.

78. The apparatus of claim 77, wherein the biological molecule is a binding member.

79. The apparatus of claim 73, further comprising an insulating material.

80. The apparatus of claim 72, wherein the biological parameter is a binding strength between a binding member and its cognate receptor.

81. The apparatus of claim 72, wherein the biological parameter is an elastic strength of a biological molecule.

82. The apparatus of claim 72, wherein the first surface has an area of less than 1000 cm².

83. An apparatus for measuring a biological parameter comprising
a first surface with a magnetic field and a magnetic field gradient comprised of a first planar, antiparallel array of electric current carrying wires,
wherein the magnetic field is one-dimensional and approximately uniform throughout an area of the first surface and decays non-linearly with distance from the first surface and wherein the first surface has an area of at least 2 cm².

84. The apparatus of claim 83, wherein the first surface has an area of at least 100 cm².

85. The apparatus of claim 83, wherein the first surface has an area of less than 1000 cm².

86. The apparatus of claim 83, further comprising a second surface having a plurality of discrete regions.

87. The apparatus of claim 86, wherein the first surface and the second surface are separated by at least 1 μm.

88. The apparatus of claim 86, wherein the second surface is conjugated to a biological molecule.

89. The apparatus of claim 88, wherein the biological molecule is a binding member.

90. The apparatus of claim 83, further comprising an insulating material.

91. The apparatus of claim 83, wherein the biological parameter is a binding strength between a binding member and its cognate receptor.

92. The apparatus of claim 83, wherein the biological parameter is an elastic strength of a biological molecule.

93. An apparatus for measuring a biological parameter comprising
a periodic magnetic array with an area of at least 2 cm² in close proximity to at least one biasing magnetic field,
wherein the biasing magnetic field is produced by a permanent magnet of area greater than the array, or by electric current carrying wires,
wherein a resultant magnetic field in the plane of the periodic magnetic array has a regular periodic variation in field strength.

94. The apparatus of claim 93, wherein the at least one permanent magnet is positioned at the side of, or below, the periodic magnetic array.

95. The apparatus of claim 93, wherein the periodic magnetic array is comprised of an first planar antiparallel array of electric current carrying wires overlaid on and perpendicular to a second planar antiparallel array of electric current carrying wires.

96. An article comprising
a one- or two-dimensional antiparallel array of magnetizations defining a surface comprised of alternating magnetic poles, the surface having an area of at least 2 cm².

97. The article of claim 96, wherein the antiparallel array of magnetizations comprises magnetic recording material.

98. The article of claim 96, wherein the antiparallel array of magnetizations comprises an array of permanent magnets.

99. The article of claim 96, wherein the antiparallel array of magnetizations comprises an array at least 50 magnets.

100. The article of claim 96, wherein the antiparallel array of magnetizations is produced by an antiparallel array of current carrying wires.

101. The article of claim 96, wherein the antiparallel array of magnetizations comprises a permanent magnet with a regularly embossed surface structure, or a magnetizable material formed in a one or two dimensional grid or with a regularly embossed surface placed in an externally applied uniform magnetic field.

102. A method for measuring a plurality of elastic strengths simultaneously comprising
contacting a first surface to a plurality of biological molecules which are labeled at one end with a magnetic particle,
applying a magnetic force to the plurality of biological molecules by positioning the first surface in proximity to a second surface having a magnetic field and a magnetic field gradient, and
determining a distance between the first surface and the magnetic particle as a measure of the elastic strength of the biological molecule after the magnetic force has been applied,
wherein the magnetic field is produced by an arrangement selected from the group consisting of an antiparallel adjacent array of permanent magnets, an antiparallel array of electric current carrying wires, a magnetic recording material with a one- or a two-dimensional periodic pattern, a permanent magnet with regularly embossed one or two dimensional surface structure, and a magnetizable material formed in a one or two dimensional grid, or with an embossed one or two dimensional surface structure in the presence of an externally applied uniform magnetic field.

103. The method of claim 102, wherein the plurality of biological molecules is comprised of different species.

104. The method of claim 102, wherein the first surface is comprised of a plurality of discrete regions.

105. The method of claim 104, wherein each of the plurality of discrete regions is conjugated to a different species of biological molecule.

106. The method of claim 104, wherein the plurality of discrete regions is at least 8.

107. The method of claim 104, wherein the area of each of the plurality of discrete regions is less than 10 mm².

108. The method of claim 104, wherein each of the plurality of discrete regions is conjugated to at least 10 biological molecules.

109. A method for determining a plurality of biological strengths simultaneously comprising
contacting a plurality of binding members conjugated to a first surface to at least one magnetically-labeled cognate receptor,
applying a magnetic force to the at least one magnetically-labeled cognate receptor by positioning the first surface in proximity to a second surface having a magnetic field and a magnetic field gradient, and
determining whether each of the plurality of binding members remains in contact with the at least one magnetically-labeled cognate receptor,
wherein the magnetic field is produced by an arrangement selected from the group consisting of an antiparallel adjacent array of permanent magnets, an antiparallel array of electric current carrying wires, a magnetic recording material with a one- or a two-dimensional periodic pattern, a permanent magnet with regularly embossed one or two dimensional surface structure, and a magnetizable material formed in a one or two dimensional grid, or with an embossed one or two dimensional surface structure in the presence of an externally applied uniform magnetic field.

110. The method of claim 109, wherein the plurality of binding members is comprised of different species.

111. The method of claim 109, wherein the first surface is comprised of a plurality of discrete regions.

112. The method of claim 111, wherein each of the plurality of discrete regions is conjugated to a different species of binding member.

113. The method of claim 111, wherein the plurality of discrete regions is at least 8.

114. The method of claim 111, wherein the area of each of the plurality of discrete regions is less than 10 mm².

115. The method of claim 111, wherein each of the plurality of discrete regions is conjugated to at least 10 binding member molecules.
